(19) United States
(12) Patent Application Publication
(10) Pub. No.: US 2009/0118509 A1
(43) Pub. Date: May 7, 2009

(54) PREPARATION OF [2-METHYL-5-PHENYL-3-(PIPERAZIN-1-YLMETHYL)] PYRROLE DERIVATIVES

(75) Inventors: Sudershan Kumar Arora, Maharashtra (IN); Neelima Sinha, Maharashtra (IN); Sanjay Jain, Maharashtra (IN); Ram Shankar Upadhayaya, Maharashtra (IN); Om Dutt Tyagi, Maharashtra (IN); Vasanth Nalam, Maharashtra (IN); Yogendra Kumar Chauhan, Maharashtra (IN)

Correspondence Address: MERCHANT & GOULD PC, P.O. BOX 2903, MINNEAPOLIS, MN, 55402-0903, US

(73) Assignee: Lupin limited, Mumbai, Maharashtra (IN)

(21) Appl. No.: 11/911,272

(22) PCT Filed: Apr. 5, 2006
(86) PCT No.: PCT/IN06/00115

§ 371 Date: Jun. 4, 2008

(30) Foreign Application Priority Data
Apr. 11, 2005 (IN) 299/KOL/2005

(51) Int. Cl. C07D 401/14

(52) U.S. Cl. 544/364

(57) ABSTRACT

A process for the preparation of compounds of Formula I and their pharmaceutically acceptable acid addition salt

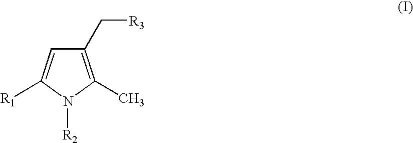

wherein,
R₁ is phenyl or substituted phenyl
R₂ is selected from a group consisting of
phenyl which is unsubstituted or substituted with 1 or 2 substituents, each independently selected from Cl, F, or
pyridine, or
naphthalene, or
NHCOR₄ wherein R₄ is aryl, unsubstituted or substituted heteroaryl, unsubstituted or substituted heterocyclyl.
R₃ is selected from a group of formula

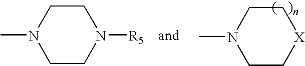

wherein R₅ is phenyl which is unsubstituted or substituted with 1 or 2 substituents each independently selected from the group consisting of halogen, C₁-C₄ alkyl, C₁-C₄ alkoxy, nitro, amino, haloalkyl, haloalkoxy etc.; unsubstituted or substituted benzyl; unsubstituted or substituted heteroaryl; unsubstituted or substituted heteroaroyl; unsubstituted or substituted diphenylmethyl,

n=0-2 and

X═—NCH₃, CH₂, S, SO, or SO₂
Such that when R₂ is phenyl, which is unsubstituted or substituted with 1 or 2 substituents, each independently selected from Cl, F; R₅ is not C₁-C₄ alkyl, or X is not —NCH₃, CH₂, S, SO, or SO₂, when n=1, or X is not —CH₂ when n=0;
comprising the steps of
(a) reacting compound of Formula II

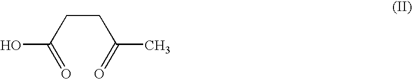

with a chlorinating agent in the presence or absence of catalytic amount of N,N-dimethylformamide to yield the compound of Formula III,

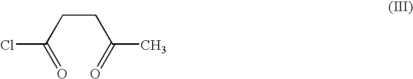

(b) reacting the compound of Formula III with a compound of Formula R₁H (R₁ is as defined above), in presence of a Lewis acid to obtain the compound of Formula IV,

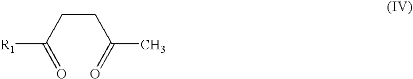

(c) reacting the compound of Formula IV with a compound of Formula R₂NH₂ (R₂ is as defined above) in presence of catalytic amounts of an aryl or alkyl sulphonic acid in an organic solvent to obtain the compound of Formula V,

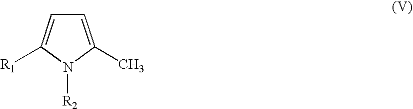

(d) reacting the compound of Formula V with various secondary amines of the Formula R₃H (R₃ is as defined above) in the presence of formaldehyde and acetic acid in acetonitrile followed by crystallization yield the compound of Formula I,

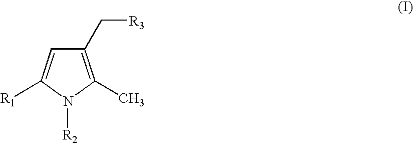

(e) purifying the compound of Formula I by crystallization,
(f) converting the purified compound of Formula I to a pharmaceutically acceptable acid addition salt.

### FIELD OF THE INVENTION

The present invention relates to an improved and industrially advantageous process for the preparation of pyrrole derivatives having antimycobacterial activity. More particularly, the present invention relates to derivatives corresponding to compounds of Formula I and their pharmaceutically acceptable acid addition salts.

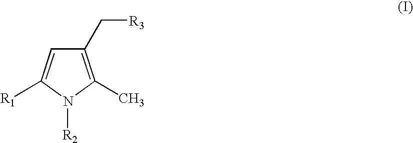

wherein,
R₁ is phenyl or substituted phenyl
R₂ is selected from a group consisting of
i) phenyl which is unsubstituted or substituted with 1 or 2 substituents, each independently selected from Cl, F, or
ii) pyridine, or
iii) naphthalene, or
iv) NHCOR₄ wherein R₄ is aryl, unsubstituted or substituted heteroaryl, unsubstituted or substituted heterocyclyl.
   R₃ is selected from a group of formula

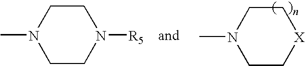

wherein R₅ is phenyl which is unsubstituted or substituted with 1 or 2 substituents each independently selected from the group consisting of halogen, C₁-C₄ alkyl, C₁-C₄ alkoxy, nitro, amino, haloalkyl, haloalkoxy etc.; unsubstituted or substituted benzyl; unsubstituted or substituted heteroaryl; unsubstituted or substituted heteroaroyl; unsubstituted or substituted diphenylmethyl,

n=0-2 and

X═—NCH₃, CH₂, S, SO, or SO₂

Such that when R₂ is phenyl, which is unsubstituted or substituted with 1 or 2 substituents, each independently selected from Cl, F; R₅ is not C₁-C₄ alkyl, or X is not —NCH₃, CH₂, S, SO, or SO₂, when n=1, or X is not —CH₂ when n=0

The compounds are useful for the treatment of tuberculosis. The preferred compound is N-{2-methyl-5-phenyl-3-[4-(3-trifluoromethyl-phenyl)-piperazin-1-ylmethyl]-pyrrol-1-yl}-isonicotinamide.

### BACKGROUND OF THE INVENTION

Tuberculosis (TB) is a contagious disease, which usually runs a protracted course, ending in death in majority of cases, with relapse being a common feature of the disease. It is one of the most important causes of prolonged disability and chronic ill health. It is caused by the tubercle bacillus Mycobacterium tuberculosis, which is comparatively difficult to control. Drugs such as isoniazid, rifampicin, pyrazinamide, ethambutol, streptomycin, p-aminosalicylic acid, ethionamide, cycloserine, capreomycin, kanamycin, thiacetazone etc. have been and are being currently used to treat TB. Among these, isoniazid, rifampicin, ethambutol and pyrazinamide are the first-line drugs of choice, which are administered either as a single drug formulation or as a fixed-dose combination of two or more of the aforesaid drugs.

Eventhough, each of the above-mentioned first-line drug regimens is highly effective for treatment of TB, they are associated with shortcomings, such as unpleasant side effects and relatively long course of treatment. The later one results in non-compliance of the patient to the treatment leading often to failure of the treatment and most importantly development of drug resistance. The development of drug resistance has long constituted a principal difficulty in treating human tuberculosis. The second-line drugs, on the other hand are less effective, more expensive and more toxic.

This underscores the importance of new antimycobacterial compounds which, (a) exhibit significantly greater antimycobacterial activity, than existing drugs, (b) provide safe and specific treatment of multi drug resistant tuberculosis (MDR TB), and (c) are useful in treatment of patients who harbour quiescent/latent tuberculosis.

Compounds of Formula I are known from PCT International Patent Application WO 2004026828, and were screened for antimycobacterial activity, in various in vitro and in vivo models in mice and guinea pigs. Several compounds exhibited strong antimycobacterial activity against sensitive and MDR strains of Mycobacterium tuberculosis in the in vitro and in vivo experiments. Further the compounds of Formula I were also found to be bioavailable, less toxic and safe compared to available anti TB drugs in various animal models.

Thus compounds of Formula I are useful for the effective treatment of Mycobacterium tuberculosis infection caused by sensitive/MDR strains. PCT International Patent Application WO 2004026828 also discloses the synthesis of compounds of Formula I,

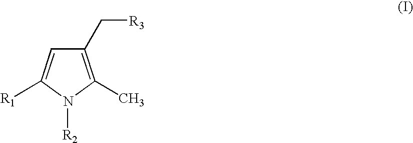

wherein,
R₁ is phenyl or substituted phenyl
R₂ is selected from a group consisting of i) phenyl which is unsubstituted or substituted with 1 or 2 substituents, each independently selected from Cl, F, or,
ii) pyridine, or
iii) naphthalene, or
iv) NHCOR₄ wherein R₄ is aryl, unsubstituted or substituted heteroaryl, unsubstituted or substituted heterocyclyl.
R₃ is selected from a group of formula

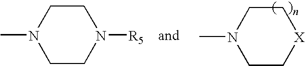

wherein R₅ is phenyl which is unsubstituted or substituted with 1 or 2 substituents each independently selected from the group consisting of halogen, C₁-C₄ alkyl, C₁-C₄ alkoxy, nitro, amino, haloalkyl, haloalkoxy etc.; unsubstituted or substituted benzyl; unsubstituted or substituted heteroaryl; unsubstituted or substituted heteroaroyl; unsubstituted or substituted diphenylmethyl,

n=0-2 and

X═—NCH₃, CH₂, S, SO, or SO₂

Such that when R₂ is phenyl, which is unsubstituted or substituted with 1 or 2 substituents, each independently selected from Cl, F; R₅ is not C₁-C₄ alkyl, or X is not —NCH₃, CH₂, S, SO, or SO₂, when n=1, or X is not —CH₂ when n=0 which comprises reacting the compound of Formula II

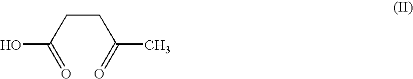

with thionyl chloride, followed by reaction with R₁H (wherein R₁ is phenyl or substituted phenyl) in presence of aluminium chloride, and then condensation with R₂NH₂ (wherein R₂ is as described above) in presence of p-toluenesulphonic acid to yield the corresponding unsubstituted pyrrole derivatives of Formula V,

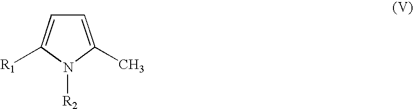

which on further treatment with suitable secondary amines in the presence of formaldehyde and acetic acid afforded the desired pyrrole derivatives of Formula I,

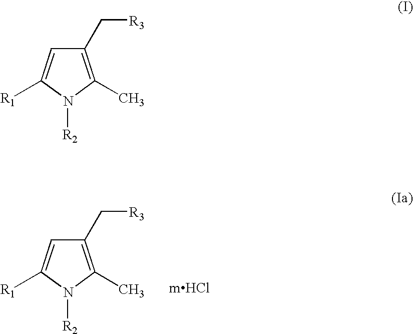

which, on reacting with hydrochloric acid give a hydrochloride salt of compound of Formula Ia.
wherein m=1-2, R₁, R₂ and R₃ are the same as defined earlier.
The above-mentioned methods in the prior art for the synthesis of compound of the Formula I suffer from the limitations,
1. In methods described in PCT International Patent Application WO 2004026828 for the synthesis of compounds of Formula I, positional isomers, the compound of Formula I′, are formed. The necessity of their removal through column chromatography decreases the yield of final pure product.

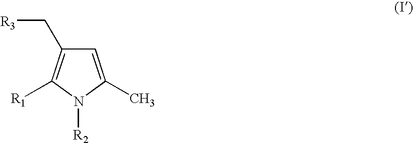

2. The synthesis of oxopentanoyl chloride (compound of Formula II) for the synthesis of compound of Formula I has been described in J. Org. Chem. 1960, 25, 390-392. It comprises reaction of levulinic acid with thionyl chloride at 50° C. for 1 h, which results in poor yield.
3. In method described in PCT International Patent Application WO 2004026828 for the synthesis of 1-aryl-pentane-1,4-dione (compound of Formula IV), impurities are formed and purification involves column chromatography which decreases the yield of the product.
4. The synthesis of the intermediate of Formula V requires the use of benzene and high temperature conditions, which involves the formation of undesired by-products.
5. The above-mentioned methods in prior art for the synthesis of all the intermediates and final compounds of Formula I involves column chromatography for purification, which is cumbersome, tedious and not practicable on an industrial scale.

### OBJECT OF THE INVENTION

The object of the present invention is to solve the problems associated with the processes for the preparation of compounds of Formula I described in the prior art and to provide an efficient process for the preparation of compounds of Formula I, and their pharmaceutically acceptable acid addition salts, thereby resulting in significant economic and technological improvement over the prior art method.

It is a further object of the present invention to provide a process that is cost effective, safe, and convenient in large scale production of compounds of Formula I.

### SUMMARY OF THE INVENTION

Thus the present invention relates to processes for the synthesis of compounds of Formula I and pharmaceutically acceptable acid addition salt thereof,

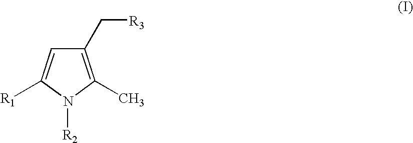

wherein
R₁ is phenyl or substituted phenyl
R₂ is selected from a group consisting of
i) phenyl which is unsubstituted or substituted with 1 or 2 substituents, each independently selected from Cl, F, or
ii) pyridine, or
iii) naphthalene, or
iv) NHCOR₄ wherein R₄ is aryl, unsubstituted or substituted heteroaryl, unsubstituted or substituted heterocyclyl.
   R₃ is selected from a group of formula

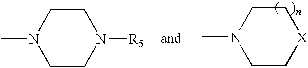

wherein R₅ is phenyl which is unsubstituted or substituted with 1 or 2 substituents each independently selected from the group consisting of halogen, C₁-C₄ alkyl, C₁-C₄ alkoxy, nitro, amino, haloalkyl, haloalkoxy etc.; unsubstituted or substituted benzyl; unsubstituted or substituted heteroaryl; unsubstituted or substituted heteroaroyl; unsubstituted or substituted diphenylmethyl,

n=0-2 and

X═—NCH₃, CH₂, S, SO, or SO₂

Such that when R₂ is phenyl, which is unsubstituted or substituted with 1 or 2 substituents, each independently selected from Cl, F; R₅ is not C₁-C₄ alkyl, or X is not —NCH₃, CH₂, S, SO, or SO₂, when n=1, or X is not —CH₂ when n=0;
which comprises:
a) reacting a compound of Formula II

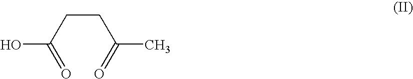

with a chlorinating agent such as thionyl chloride, phosphorous halide in the presence or absence of catalytic amount of N,N-dimethylformamide to yield the organic compound of Formula III,

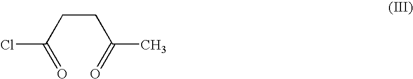

b) reacting the resulting compound of formula III with compound of Formula R₁H (R₁ is defined as above), in presence of a Lewis acid such as aluminum chloride to yield organic compound of Formula IV

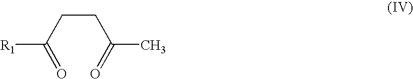

wherein R₁ is the same as defined earlier.
c) reacting the compound of Formula IV with compound of Formula R₂NH₂ (R₂ is defined above) in the presence catalytic amounts of aryl or alkyl sulphonic acids such as p-toluenesulphonic acid, benzene sulphonic acid or methane sulphonic acid in organic solvents selected from chlorinated hydrocarbon such as dichloromethane, dichloroethane, chloroform, or lower alkyl substituted or un-substituted aromatic hydrocarbon solvents such as benzene, toluene or xylene to yield the compound of Formula V

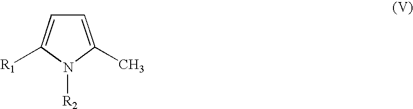

d) reacting compound of Formula V with various secondary amines of the Formula R₃H in the presence of formaldehyde and acetic acid in acetonitrile to give the compound of Formula I

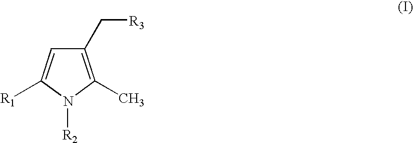

e) purifying the compound of Formula I obtained above by crystallization from a mixture of solvents such as ethyl acetate-hexane, ethyl acetate-isopropyl alcohol or ethyl acetate-cyclohexane to remove the compound of Formula I′, the positional isomer of the compound of Formula I.

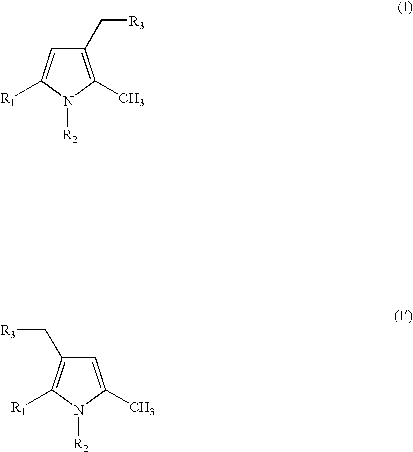

wherein R₁, R₂ and R₃ are same as defined earlier,
f) converting the compound of formula I obtained in step (e) to a pharmaceutically acceptable salt, for example, treating with hydrochloric acid in an organic solvent such as ethyl alcohol, ethyl acetate, isopropnol, diethyl ether or diisopropyl ether to yield the hydrochloride salt of compound of Formula I

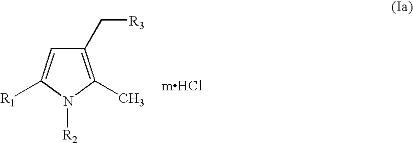

wherein m=1-2, R₁, R₂ and R₃ are the same as defined earlier.

The present inventors have found that the separation of the isomeric compound of Formula I′ from the compound of Formula I can be achieved by one or more crystallization from a mixture of solvents such as ethyl acetate-hexane, ethyl acetate-isopropyl alcohol or ethyl acetate-cyclohexane thereby obviating the need for column chromatography.

The inventors have further found that the yield and purity of the chlorination of the compound of formula II can be improved by conducting the reaction in the presence of catalytic amount of N,N-dimethylformamide or controlled temperature conditions.

The inventors have also found that the formation of pyrrole ring by the cyclization of the 1,4-dione compound of the formula IV with an amine of formula R₂NH can be facilitated by the use of catalytic amounts of aryl or alkyl sulphonic acids and in solvents such as chlorinated hydrocarbon or aromatic hydrocarbon.

### DETAILED DESCRIPTION OF THE INVENTION

The present invention relates to processes for the syntheses of compounds of Formula I and their pharmaceutically acceptable salts, preferably, the hydrochloride salt.

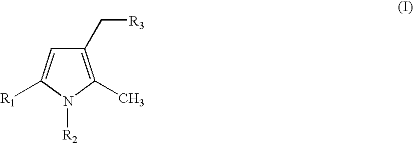

wherein,
R₁ is phenyl or substituted phenyl
R₂ is selected from a group consisting of
i) phenyl which is unsubstituted or substituted with 1 or 2 substituents, each independently selected from Cl, F, or
ii) pyridine, or
iii) naphthalene, or
iv) NHCOR₄ wherein R₄ is aryl, unsubstituted or substituted heteroaryl, unsubstituted or substituted heterocyclyl.
   R₃ is selected from a group of formula

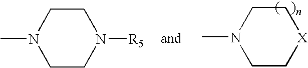

wherein R₅ is phenyl which is unsubstituted or substituted with 1 or 2 substituents each independently selected from the group consisting of halogen, C₁-C₄ alkyl, C₁-C₄ alkoxy, nitro, amino, haloalkyl, haloalkoxy etc.; unsubstituted or substituted benzyl; unsubstituted or substituted heteroaryl; unsubstituted or substituted heteroaroyl; unsubstituted or substituted diphenylmethyl,

n=0-2 and

X═—NCH₃, CH₂, S, SO, or SO₂

Such that when R₂ is phenyl, which is unsubstituted or substituted with 1 or 2 substituents, each independently selected from Cl, F; R₅ is not C₁-C₄ alkyl, or X is not —NCH₃, CH₂, S, SO, or SO₂, when n=1, or X is not —CH₂ when n=0; which comprises:
a) reacting a compound of Formula II

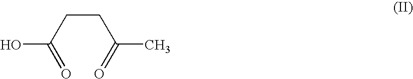

with chlorinating agent such as thionyl chloride, phosphorous halides. Preferably, the chlorinating agent is thionyl chloride.

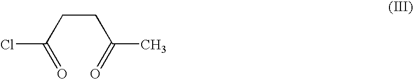

b) reacting the compound of Formula III with compound of Formula R₁H (R₁ is defined as above), in presence of a Lewis acid such as aluminum chloride to yield the compound of Formula IV

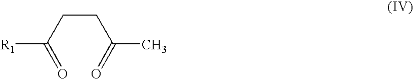

wherein R₁ is the same as defined earlier.
c) reacting the compound of Formula IV with compound of Formula R₂NH₂ (R₂ is defined above) in the presence of catalytic amount of aryl or alky sulphonic acid such as p-toluenesulphonic acid, benzene sulphonic acid or methane sulphonic acidin organic solvents selected from chlorinated hydrocarbon such as dichloromethane, dichloroethane, chloroform, or lower alkyl substituted or un-substituted aromatic hydrocarbon solvents such as toluene, xylene or benzene and a mixture thereof to yield the compound of Formula V

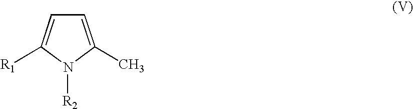

d) reacting the compound of formula V with various secondary amines of the Formula R₃H in the presence of formaldehyde and acetic acid in a organic solvent such as acetonitrile to obtain the compound of formula I.

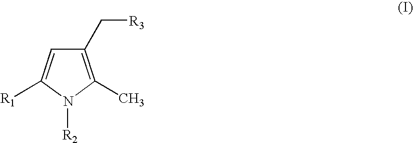

e) purifying the compound of formula I obtained above by crystallization from a mixture of solvents such as ethyl acetate-hexane, ethyl acetate-isopropyl alcohol or ethyl acetate-cyclohexane to remove the trace amount of compound of Formula I′, the positional isomer of the compound of Formula I.
f) reacting the compound of Formula I obtained above in step (e) with hydrochloric acid in organic solvents such as ethyl alcohol, ethyl acetate, isopropanol, diethyl ether or diisopropyl ether to yield the hydrochloride salt of the compound of Formula I

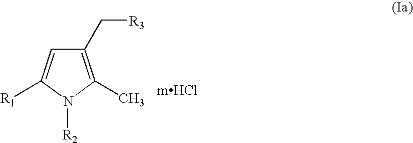

wherein m=1-2, R₁, R₂ and R₃ are the same as defined earlier.

The chlorinating agent is used in an amount of 1 to 1.2 equivalents.

The chlorination using thionyl chloride is carried out in presence or absence of catalytic amount of N,N-dimethylformamide (DMF). The chlorination in presence of DMF is carried out between 10-40° C., preferably between 20-30° C. In the absence of DMF, the chlorination is carried out between 40-60° C., preferably between 50-55° C. to yield the organic compound of Formula III.

The crystallization removes the positional isomer of formula I′ formed in about 15-20%.

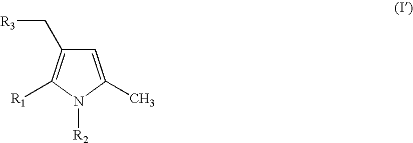

wherein R₁, R₂ and R₃ are same as defined earlier,
The crystallization may be repeated one or more times to obtain the compound of Formula I having purity >99%.

The overall yield of compounds of Formula I by the process described herein is increased to about 11.3% against about 2.6% as reported in PCT International Patent Application WO 2004026828.

Particular example of the compound synthesized according to the process of present invention and capable of being produced by the above mentioned process include:
N-{2-methyl-5-phenyl-3-[4-(3-trifluoromethyl-phenyl)-piperazin-1-ylmethyl]-pyrrol-1-yl}-isonicotinamide

The preferred compound produced according to the process of present invention is N-{2-methyl-5-phenyl-3-[4-(3-trifluoromethyl-phenyl)-piperazin-1-ylmethyl]-pyrrol-1-yl}-isonicotinamide.

The isomer B is separated from A by one or more recrystallization from a mixture of ethyl acetate and cyclohexane.

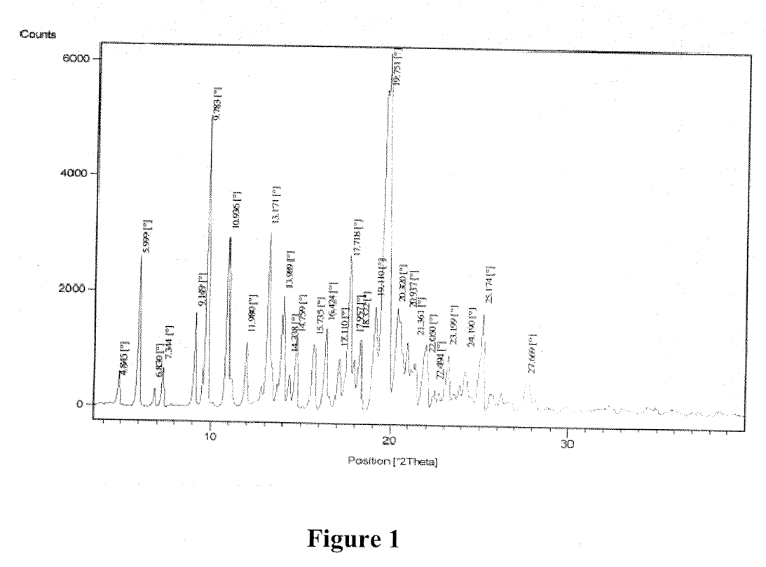

The free base N-{2-methyl-5-phenyl-3-[4-(3-trifluoromethyl-phenyl)-piperazin-1-ylmethyl]-pyrrol-1-yl}-isonicotinamide is crystalline as evident from the characteristic powder X-ray diffraction pattern given in FIG. 1 having 2θ values 4.85, 5.99, 6.83, 7.34, 9.15, 9.78, 10.93, 11.98, 13.17, 13.98, 14.33, 14.75, 15.73, 16.42, 17.11, 17.72, 17.95, 18.32, 19.11, 19.75, 20.32, 21.36, 22.04, 23.19, 25.17

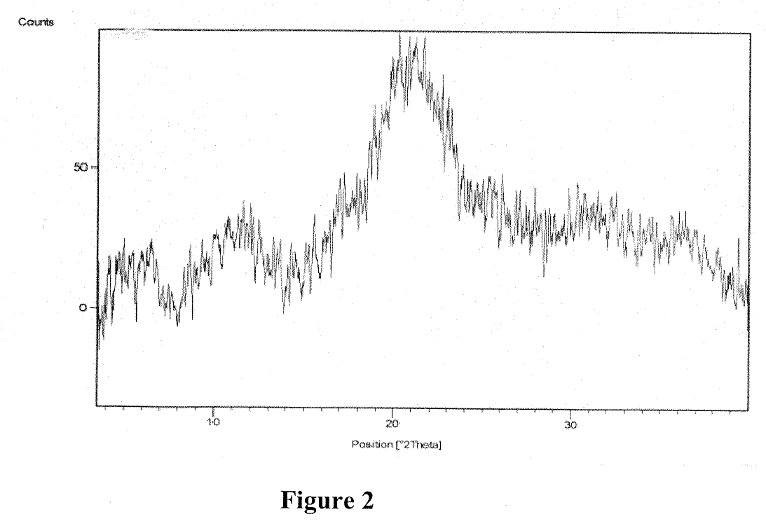

The free base N-{2-methyl-5-phenyl-3-[4-(3-trifluoromethyl-phenyl)-piperazin-1-ylmethyl]-pyrrol-1-yl}-isonicotinamide is treated with HCl to obtain the dihydrochloride salt in an amorphous form as characterized by the powder X-ray diffraction pattern given in FIG. 2. In the following section particular embodiments are described in a way to illustrate the process of invention. However, this is not intended in any way to limit the scope of the present invention. While the present invention has been described in terms of its specific embodiments, certain modifications and equivalents will be apparent to those skilled in the art and are intended to be included within the scope of the present invention.

### EXAMPLE 1

### Preparation of N-{2-methyl-5-phenyl-3-[4-(3-trifluoromethyl-phenyl)-piperazin-1-ylmethyl]-pyrrol-1-yl}-isonicotinamide hydrochloride

### Step (a): Preparation of 4-oxo-pentanoyl Chloride

To a stirred mixture of levulinic acid (340.23 g, 2.93 mol) and N,N-dimethylformamide (6.8 mL, catalytic amount) was added thionyl chloride (367.36 g, 3.087 mol, 1.05 equivalent) drop-wise at 20-30° C. in 1.5-2.0 h. After the complete addition of thionyl chloride, the reaction mixture was stirred at same temperature for 0.5 h (completion of reaction or formation of acid chloride was monitored by GC). After the completion of reaction, thionyl chloride was distilled off under reduced pressure at 20-30° C. Traces of thionyl chloride were removed by adding benzene (136 mL) under reduced pressure at 30-35° C. and residue was dried at reduced pressure (1-2 mm) at 20-30° C. for 30-60 min to yield 370 g (93.8%) of 4-oxo-pentanoyl chloride as light orange oil.

### Step (b): Preparation of 1-phenyl-pentane-1,4-dione

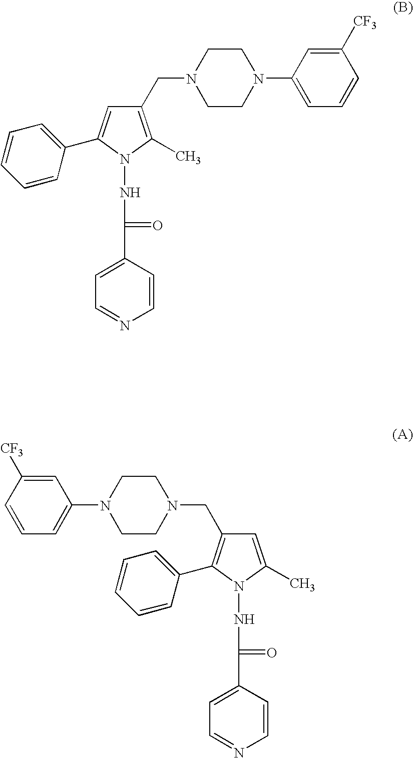

To a stirred suspension of benzene (3700 mL, 10 T w/v of acid chloride) and anhydrous aluminium chloride (440.02 g, 3.30 mol, 1.20 equivalent) was added 4-oxo-pentanoyl chloride (370 g, 2.75 mol) drop-wise; the rate of addition was regulated so that the addition required 1.5-2 h and the temperature of the reaction mixture was kept at 25-35° C. The reaction was completed in 2 h and monitored by GC. After completion of reaction, the reaction mixture was added slowly into cold (5-10° C.) 5% HCl (3700 mL) solution maintaining the temperature below 30° C. The layers were separated; aqueous layer was extracted with ethyl acetate (1×1850 mL). The combined organic phase was washed with water (1×1850 mL), 5% NaHCO₃ solution (1×1850 mL), water (1×1850 mL), 5% NaCl solution (1×1850 mL), dried (Na₂SO₄), filtered and concentrated under reduced pressure at 35-40° C., which was finally dried under reduced pressure (1-2 mm) at 35-40° C. to yield 185.6 g (38.3%) of 1-phenyl-pentane-1,4-dione as thick oil.

### Step (c): Preparation of N-(2-methyl-5-phenyl-pyrrol-1-yl)-isonicotinamide

A mixture of 1-(phenyl)-pentane-1,4-dione (185 g, 1.05 mol), isonicotinic hydrazide (158.4 g, 1.155 mol, 1.1 equivalent), p-toluenesulphonic acid (1.85 g, 1% w/w) and dichloromethane (1850 mL) was heated under reflux at 40-50° C. under azeotropic distillation for 2-3 h (water was collected in dean stark apparatus). The completion of reaction was monitored by HPLC. After cooling to 25-30° C. the resulting mixture was washed with saturated NaHCO₃ solution (1×925 mL), aqueous layer was back extracted with EtOAc (1×925 mL). The combined organic layers were washed with water (1×925 mL), 5% brine solution (1×925 mL), dried (Na₂SO₄) and filtered. The filtrate was concentrated under reduced pressure to obtain the solid product, which was further dried under reduced pressure (1-2 mm) at 35-40° C. To this, cyclohexane (925 mL) was added and stirred for 25-30 min, solid separated out was filtered washed with cyclohexane (370 mL). This process was repeated two times more with the same amount of cyclohexane and finally solid was dried under reduced pressure (1-2 mm) at 40-50° C.; yield 162.23 g (55.7%).

White solid, mp 177-179° C. ¹H NMR (CDCl₃): δ 2.10 (s, 3H), 5.98 (d, J=3.4 Hz, 1H), 6.22 (d, J=3.7 Hz, 1H), 7.23-7.28 (m, 5H), 7.50 (d, J=5.6 Hz, 2H), 8.55 (d, J=5.0 Hz, 2H), 9.82 (s, 1H). MS: m/z (%) 278 (100) [M+1]. Anal. Calcd for C₁₇H₁₅N₃O (277.32): C, 73.63; H, 5.45; N, 15.15. Found: C, 73.92; H, 5.67; N, 15.29.

### Step (d): Preparation of N-{2-methyl-5-phenyl-3-[4-(3-trifluoromethyl-phenyl)-piperazin-1-ylmethyl]-pyrrol-1-yl}-isonicotinamide

To a stirred solution of N-(2-methyl-5-phenyl-pyrrol-1-yl)-isonicotinamide (160 g, 0.577 mol) in acetonitrile (1600 mL), was added drop-wise through pressure equalizing funnel a mixture of 1-(3-trifluoromethyl-phenyl)-piperazine monohydrochloride (153.75 g, 0.667 mol, 1.155 equivalent), formaldehyde (17.34 g, 0.577 mol, 1.0 equivalent) and acetic acid (480 mL) at 25-30° C. over a period of 60-90 min. The resulting reaction mixture was stirred for 14-16 h at same temperature and completion of reaction was monitored by TLC. After the completion of reaction, reaction mixture was treated with 20% aqueous NaOH solution (2600 mL). Layers were separated, EtOAc (4000 mL) was added to organic layer, washed with water (2×2000 mL), brine (2×1250 mL), dried (Na₂SO₄), and filtered. The filtrate was concentrated under reduced pressure at 35-38° C. and then dried under reduced pressure (1-2 mm) to yield the mixture of N-{5-methyl-2-phenyl-3-[4-(3-trifluoromethyl-phenyl)-piperazin-1-ylmethyl]-pyrrol-1-yl}-isonicotinamide (A) and N-{2-methyl-5-phenyl-3-[4-(3-trifluoromethyl-phenyl)-piperazin-1-ylmethyl]-pyrrol-1-yl}-isonicotinamide (B), yield 289 g (97.8%). The ratio of A and B was determined by reverse phase HPLC, which was found to be 19.4% and 76.7%, respectively.

### Step (e): Purification of N-{2-methyl-5-phenyl-3-[4-(3-trifluoromethyl-phenyl)-piperazin-1-ylmethyl]-pyrrol-1-yl}-isonicotinamide

i) The mixture of A and B obtained from Step (d) (279 g) was dissolved in EtOAc (1960 mL, 7 times) by heating at 50-60° C. To this activated charcoal (14 g) was added and stirred for 10 min at the same temperature, filtered the activated charcoal through celite bed at 50-60° C., washed with EtOAc (560 mL). After cooled to 25-30° C., cyclohexane (2800 mL) was added to the filtrate and stirred the reaction mixture for 14-15 h at 20-35° C. Solid separated out was filtered, washed with cyclohexane (3500 mL) and dried under reduced pressure (1-2 mm) for 4-5 hours. Yield 151 g (52%). Ratio of A and B was found to be 1.7% and 96.6%, respectively.

ii) The mixture of A and B obtained from Step (e)(i) (151 g) was dissolved in EtOAc (755 mL, 5 times) by heating at 50-60° C. After cooled to 25-30° C., cyclohexane (1510 mL) was added and stirred the reaction mixture for 14-15 h at 20-35° C. Solid separated out was filtered, washed with cyclohexane (3000 mL) and dried under reduced pressure (1-2 mm) for 4-5 hours. Yield 140 g (92%). Ratio of A and B was found to be 0.2% and 98.1%, respectively.

Off white solid, mp 191-193° C. ¹H NMR (CDCl₃): δ 2.13 (s, 3H), 2.60 (br s, 4H), 3.13 (br s, 4H), 3.41 (s, 2H), 6.24 (s, 1H), 6.97-7.29 (m, 9H), 7.53 (d, J=5.6 Hz, 2H), 8.50 (s, 1H), 8.70 (d, J=5.6 Hz, 2H). ¹³C NMR (CDCl₃): δ 165.93, 151.77, 150.86, 139.74, 133.02, 131.99, 131.43, 129.92, 129.01, 127.79, 127.49, 121.74, 119.09, 116.18, 115.05, 112.48, 109.51, 54.87, 52.99, 48.93, 9.77. MS: m/z (%) 520 (100) [M+1]. Anal. Calcd for C₂₉H₂₈F₃N₅O (519.56): C, 67.04; H, 5.43; N, 13.48. Found: C, 67.36; H, 5.71; N, 13.69.

The free base N-{2-methyl-5-phenyl-3-[4-(3-trifluoromethyl-phenyl)-piperazin-1-ylmethyl]-pyrrol-1-yl}-isonicotinamide is obtained in a crystalline form having characteristic powder X-ray diffraction pattern given in FIG. 1 with 2θ values 4.85, 5.99, 6.83, 7.34, 9.15, 9.78, 10.93, 11.98, 13.17, 13.98, 14.33, 14.75, 15.73, 16.42, 17.11, 17.72, 17.95, 18.32, 19.11, 19.75, 20.32, 21.36, 22.04, 23.19, 25.17

### Step (f): Preparation of N-{2-methyl-5-phenyl-3-[4-(3-trifluoromethyl-phenyl)-piperazin-1-ylmethyl]-pyrrol-1-yl}-isonicotinamide hydrochloride

To a stirred solution of 6% w/v HCl-EtOAc solution (821.8 mL, 1.351 mol, 7.0 equivalent) in EtOAc (2000 mL) was added a solution of N-{2-methyl-5-phenyl-3-[4-(3-trifluoromethyl-phenyl)-piperazin-1-ylmethyl]-pyrrol-1-yl}-isonicotinamide (100 g, 0.193 mol) in EtOAc (2000 mL) through dropping funnel at 15-20° C. When the addition was completed (˜60 min), the reaction mixture was stirred at 10-15° C. for 1 h and then nitrogen gas was passed through reaction mass for 1 h until all the excess HCl fumes were removed. Solid so obtained was filtered through suction in an inert atmosphere, washed with ethyl acetate (2×500 mL), diisopropyl ether (2×500 mL) and dried in vacuum oven under reduced pressure (1-2 mm) at 35-40° C. for 15-20 h. Yield 115 g (99%).

Yellow solid, mp 177-179° C. ¹H NMR (DMSO-d₆): δ 2.21 (s, 3H), 3.11-3.42 (m, 6H), 3.93-4.23 (m, 4H), 6.62 (s, 1H), 7.09-7.51 (m, 9H), 8.19-8.21 (d, 2H, J=4.6 Hz), 8.95-8.97 (d, 2H, J=4.6 Hz), 11.30 (br s, 1H), 12.86 (s, 1H). MS: m/z (%) 520 (100) [M+1]. Anal. Calcd for C₂₉H₂₈F₃N₅O₂HCl.3H₂O (646.53): C, 53.87; H, 5.61; N, 10.83. Found: C, 53.67; H, 5.59; N, 10.86.

The product obtained was amorphous in nature having the characteristic X-ray powder diffraction pattern given in FIG. 2.

1. A process for the preparation of compounds of Formula I and their pharmaceutically acceptable acid add addition salt 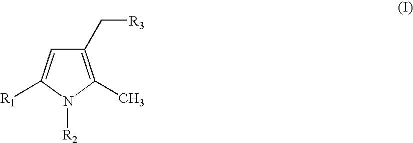
wherein,
R₁ is phenyl or substituted phenyl
R₂ is selected from a group consisting of phenyl which is unsubstituted or substituted with 1 or 2 substituents, each independently selected from Cl, F, or
pyridine, or
naphthalene, or
NHCOR₄ wherein R₄ is aryl, unsubstituted or substituted heteroaryl, unsubstituted or substituted heterocyclyl.
R₃ is selected from a group of formula 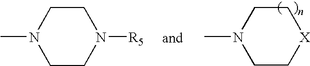
wherein R₅ is phenyl which is unsubstituted or substituted with 1 or 2 substituents each independently selected from the group consisting of halogen, C₁-C₄ alkyl, C₁-C₄ alkoxy, nitro, amino, haloalkyl, haloalkoxy etc.; unsubstituted or substituted benzyl; unsubstituted or substituted heteroaryl; unsubstituted or substituted heteroaroyl; unsubstituted or substituted diphenylmethyl,
n=0-2 and
X═—NCH₃, CH₂, S, SO, or SO₂
Such that when R₂ is phenyl, which is unsubstituted or substituted with 1 or 2 substituents, each independently selected from Cl, F; R₅ is not C₁-C₄ alkyl, or X is not —NCH₃, CH₂, S, SO, or SO₂, when n=1, or X is not —CH₂ when n=0;
comprising the steps of
(a) reacting compound of Formula II 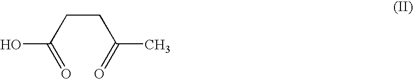
with a chlorinating agent like thionyl chloride optionally in the presence of catalytic amount of N,N-dimethylformamide to yield the compound of Formula III, 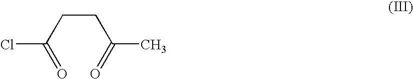
(b) reacting the compound of Formula III with a compound of Formula R₁H (R₁ is as defined above), in presence of a Lewis acid to obtain the compound of Formula IV, 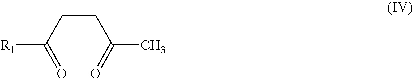
(c) reacting the compound of Formula IV with a compound of Formula R₂NH₂ (R₂ is as defined above) in presence of catalytic amounts of p-toluenesulphonic acid in an organic solvent is selected from dichloromethane, dichloroethane, chloroform, benzene, toluene, xylene and mixtures thereof. to obtain the compound of Formula V, 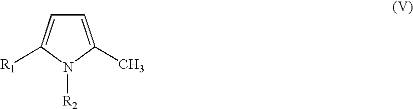
(d) reacting the compound of Formula V with various secondary amines of the Formula R₃H (R₃ is as defined above) in the presence of formaldehyde and acetic acid in acetonitrile followed by crystallization yield the compound of Formula I, 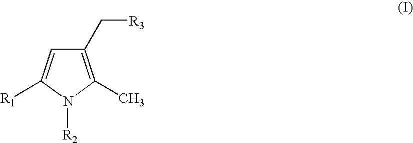
(e) purifying the compound of Formula I by crystallization in a mixture of organic solvents, wherein the mixture of organic solvent is selected from ethyl acetate-hexane, ethyl acetate-cyclohexane, and isopropyl alcohol-hexane,
(f) converting the purified compound of Formula I to a pharmaceutically acceptable acid addition salt.

2. The process of claim 1, wherein the compound of Formula I is N-{2-methyl-5-phenyl-3-[4-(3-trifluoromethyl-phenyl)-piperazin-1-ylmethyl]-pyrrol-1-yl}-isonicotinamide hydrochloride.

3. The process of claim 1, wherein the chlorinating agent is selected from thionyl chloride and phosphorous halides.

4. The process of claim 1, wherein the chlorination of compound of Formula II with thionyl chloride is carried out at a temperature ranging from 20-30° C.

5. The process of claim 1, wherein the chlorination of the compound of Formula II with thionyl chloride is carried out without N,N-dimethylformamide.

6. The process of claim 5, wherein the reaction of compound of Formula II with thionyl chloride is carried out at temperature ranging from 50-55° C.

7. The process of claim 1, wherein the reaction of compound of Formula III with R₁H (R₁ is defined as above) is carried out in the presence of Lewis acid aluminium chloride.

8. The process of claim 1 wherein the reaction of compound of Formula IV with R₂NH₂ (R₂ is defined as above) is carried out at a temperature ranging from 40-140° C.

9. The process of claim 1, wherein the reaction of compound of Formula V with compound of formula R₃H (R₃ is defined as above) is carried out in the presence of formaldehyde and acetic acid in acetonitrile.

10. The process of claim 9, wherein the reaction of compound of Formula V with R₃H(R₃ is defined as above) is carried out at a temperature ranging from 20-30° C.

11. The process of claim 1, wherein the purified compound of Formula I is converted to its hydrochloride salt of Formula Ia by treating with hydrochloric acid in an organic solvent selected from dichloromethane, ethyl acetate, ethanol and diethyl ether and mixtures thereof. 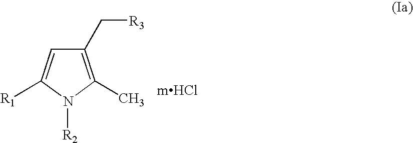
wherein m=1-2,
R₁, R₂ and R₃ are the same as defined earlier.

12. A crystalline form of the compound N-{2-methyl-5-phenyl-3-[4-(3-trifluoromethyl-phenyl)-piperazin-1-ylmethyl]-pyrrol-1-yl}-isonicotinamide of formula I having characteristic powder X-ray diffraction pattern with 2θ values at 4.85, 5.99, 6.83, 7.34, 9.15, 9.78, 10.93, 11.98, 13.17, 13.98, 14.33, 14.75, 15.73, 16.42, 17.11, 17.72, 17.95, 18.32, 19.11, 19.75, 20.32, 21.36, 22.04, 23.19, 25.17.
